(19) United States
(12) Patent Application Publication
(10) Pub. No.: US 2024/0196893 A1
(43) Pub. Date: Jun. 20, 2024

(54) LOW TOXICITY COMPOUNDS FOR USE AS INSECTICIDES AND METHOD OF PRODUCING SAID COMPOUNDS

(71) Applicants: UNIVERSIDADE DO PORTO, Porto (PT); UNIVERSIDADE DO MINHO, Braga (PT)

(72) Inventors: David Alexandre MICAEL PEREIRA, Porto (PT); Maria DO SAMEIRO TORRES GONÇALVES, Braga (PT); António Belmiro GIL SILVA FORTES, Braga (PT); Elisabete Maria DOS SANTOS CASTANHEIRA COUTINHO, Braga (PT); Renato Joel BARROS PEREIRA, Porto (PT)

(21) Appl. No.: 18/556,474

(22) PCT Filed: Apr. 26, 2021
(86) PCT No.: PCT/IB2021/053422

§ 371 Date: Oct. 20, 2023

(30) Foreign Application Priority Data
Apr. 20, 2021 (PT) 117184

(51) Int. Cl. A01N 37/44; A01P 7/04; C07C 229/56; C07C 229/60

(52) U.S. Cl. CPC A01N 37/44; A01P 7/04; C07C 229/56; C07C 229/60

(57) ABSTRACT

The present application relates to a compound of Formula (I) and its analogues, which are obtained by chemical synthesis. The disclosed compounds are suitable to be used as insecticides by killing insect cells in a selective fashion, namely by not being toxic to human cells. These compounds are different from existing insecticide solutions given their potency, selectivity and non-toxicity for human cells, and mechanism of action via caspase activation.

### TECHNICAL FIELD

This application relates to a compound of Formula (I), and its analogues, with low human toxicity for use as insecticide and a method to produce said compound of Formula (I) and its analogues.

### BACKGROUND ART

According to Food and Agriculture Organization of the United Nations (FAO), food demand in the USA alone is expected to increase from 50% to 90% by the year 2050, a trend that fallows the projected growth in population. For this reason, pesticides, specifically insecticides, will also grow in demand in order to address the increasing food production.

The current global market size for pesticides is estimated at 14.5 billion USD in 2017, being projected to grow at Compound Annual Growth Rate (CAGR) of 5.8%, thus reaching 19.3 billion by 2022¹. The current largest market is Asia Pacific, which is also the fastest growing market.

In the specific case of Europe, the insecticides market is expected to grow at a CAGR of 5.3% and reach USD 5.88 billion in 2025, thus departing from the 2020 value of USD 4.77 billion².

There has been increasing research towards the discovery of new insecticides that have an adequate safety profile while maintaining the potency of classic insecticides, such as organophosphates and pyrethrins.

There are currently no scientific papers, patents or other sources of information reporting or addressing the insecticide activity of the disclosed compounds or their analogues. Some patents refer the disclosed compound, however the activities described are unrelated.

Previous methods reported in literature include reaction of dodecanamine with 2-chlorobenzoic acid using dimethylformamide (DMF) and copper powder using precautions to exclude moisture from reaction; alternatively, the desired compound was also prepared using the same substrates, but through an ultrasound-promoted reaction in DMF at room temperature³.

In 2009, it has been reported a method for the synthesis of the same compound, using dodecylamino hydrochloride and 2-bromobenzoic acid as substrates under copper catalysis using bifenilnaftol (BINOL) as ligand in DMF at room temperature⁴. Another report describes the reaction of 1-chlorododecane with anthranilic acid in the presence of anion exchange resin in OH form⁵.

Compared with these previous methods, the present methodology uses 2-aminobenzoic acid or 3-aminobenzoic acid and 1-chloroalkane or 1-bromodoalkane as substrates in an alcohol solvent, and no catalyst is needed.

The state of the art in the field involves the use of insecticides that belong to classes such as carbamates and organophosphates, which are acetylcholinesterase inhibitors, organochlorides, which are GABA-gated channel antagonists, and pyrethroids, which are sodium channel modulators.

The compound of Formula (I) disclosed herein presents a different mechanism of action, namely direct activation of cell death via caspase activation.

### SUMMARY

The present application relates to a compound of Formula (I) and its analogues for use as insecticide, wherein Formula (I) is:

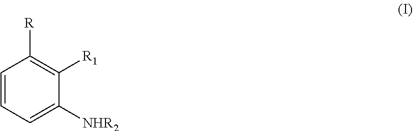

wherein R and R1 are selected from hydrogen or carboxyl group; when R is hydrogen, R1 is a carboxyl group; and when R is a carboxyl group, R1 is hydrogen;
and R2 is an alkyl group substituted or unsubstituted.

In one embodiment the alkyl group comprises 12 to 20 carbon atoms.

In another embodiment the alkyl group comprises 12 to 15 carbon atoms.

In yet another embodiment the alkyl group is selected from dodecyl, tridecyl and pentadecyl groups, substituted or unsubstituted.

In one embodiment the compound is a 2-(dodecylamino) benzoic acid of Formula (II):

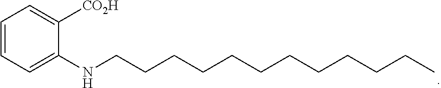 [SMILES: CCCCCCCCCCCCNc1ccccc1C(=O)O]

In another embodiment the compound is a 3-(dodecylamino)benzoic acid of Formula (III):

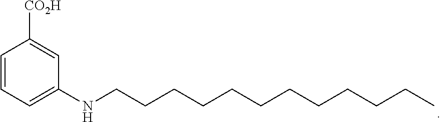 [SMILES: CCCCCCCCCCCCNc1cccc(C(=O)O)c1]

The present application also relates to a formulation for use as insecticide, comprising a compound of Formula (I) or its analogues.

The present application further relates to a method of producing the compound of Formula (I) and its analogues comprising the following steps:
Adding a solution of 1-chloroalkene or 1-bromoalkene to a solution of 2-aminobenzoic acid or of 3-aminobenzoic acid;
Heating the reaction mixture at a temperature between 50° C. and 70° C., using an alcohol as solvent, and with a final concentration of 0.05 to 0.2 M of the reaction mixture, for a time period between 24 to 64 hours;
Evaporating the solvent under reduced pressure.

In one embodiment the solution of 1-chloroalkene or of 1-bromoalkene has a concentration between 0.1 to 0.4 M.

In another embodiment the solution of 2-aminobenzoic acid or of 3-aminobenzoic acid has a concentration between 0.05 to 0.2 M.

In another embodiment the alcohol solvent is selected from methanol or ethanol in a concentration from 80 to 97% (w/w).

In yet another embodiment the evaporation step is performed at a temperature between 20 and 70° C.

### DETAILED DESCRIPTION

The present application relates to compounds related to ginkgolic acids. The presently disclosed compound of Formula (I) and its analogues are suitable for use as insecticide.

The compounds of the present application can be synthesized using less expensive reagents, by a single straightforward reaction from the commercial reagents with yield higher than 60%.

The compound and its analogues of the present application for use as insecticide have the Formula (I):

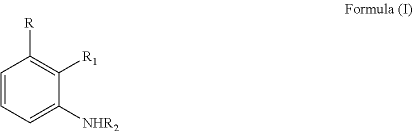

wherein R and R1 are selected from hydrogen or carboxyl group;
when R is hydrogen, R1 is a carboxyl group; and when R is a carboxyl group, R1 is hydrogen,
and R2 is an alkyl group substituted or unsubstituted.

In the present application, the term “analogues” is understood as compounds containing the benzoic acid skeleton, plus the amine group bearing an alkyl chain with different number of methylenic groups.

In the present application, the term “alkyl” relates to a linear hydrocarbon group comprising from 12 to 20 carbon atoms, preferably 12 and 15 carbon atoms. For example, the alkyl group can be selected from the dodecyl, tridecyl and pentadecyl groups, substituted or unsubstituted.

In one embodiment, the compound of Formula (I) and its analogues relate to compounds known as 2-(alkylamino) benzoic acids and 3-(alkylamino) benzoic acids suitable for use as insecticide.

In one embodiment, the compound of Formula (I) includes the 2-(dodecylamino) benzoic acid (6a in FIG. 1). The compound 6a derives from Formula (I), where R=H, R₁=CO₂H and R₂=(CH₂)₁₁CH₃.

The disclosed compound (6a) has the following Formula (II):

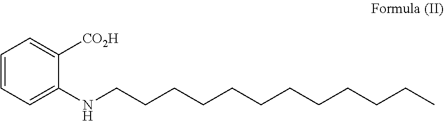 [SMILES: CCCCCCCCCCCCNc1ccccc1C(=O)O]

In one embodiment, the compound of Formula (I) includes the 3-(dodecylamino)benzoic acid (6b in FIG. 1), known as Formula (III). The compound 6b derives from Formula (I), where R=CO₂H, R₁=H and R₂=(CH₂)₁₁CH₃.

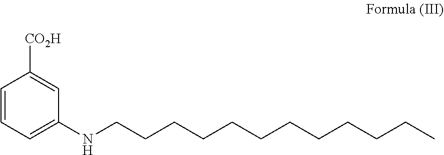 [SMILES: CCCCCCCCCCCCNc1cccc(C(=O)O)c1]

A compound of Formula (I) and its analogues are synthesized by N-alkylation reaction of the amino group of 2-aminobenzoic and 3-aminobenzoic acids in the presence of the corresponding alkyl bromide or chloride using an alcohol as solvent.

The solvents used are alcohols such as methanol or ethanol, and the reaction occurs under heating at atmospheric pressure.

After the synthesis, removal of the solvent from the reaction mixture is extremely easy by evaporation at mild temperature and pressure conditions, due to the low boiling point of the solvents used.

The synthesis of the compounds occurs through a single chemical reaction between only two reagents, without the need of base or catalyst, nor any intermediate step (derivatization, protection or deprotection), which facilitates the process of obtaining the required compounds, decreases the production cost and their impact on the environment.

The compound (6a) disclosed is more potent than the commercial insecticide chlorpyrifos (O,O-diethyl O-(3,5,6-trichloropyridin-2-yl) phosphorothioate), namely by killing more than the triple of insect cells at the same concentration of the commercial benchmark.

The disclosed compound (6a) has no toxicity in human cells, contrarily to the ginkgolic acid, 2-(heptadec-10-en-1-yl) -6-hydroxybenzoic acid (1), or the synthetic commercial insecticide evaluated, chlorpyrifos.

The disclosed compound of Formula (I) and its analogues presents a mechanism of action distinct of the commercial insecticide chlorpyrifos, which can be of interest as a strategy to overcome pesticide resistances. Specifically, the compound (6a) of Formula (II), is capable of activating effector caspases of the DRICE (Death related ICE) family on insect cells, which are involved in cellular cell death, while the commercial insecticide is unable to trigger this effect. It is expected that all compounds derived from compound of Formula (I) and its analogues have the same absence of toxicity in human cells and are capable of activating effector caspases of the DRICE family on insect cells.

This technology can result in a product, which can be commercialized to be used as an insecticide, either as a stand-alone or in conjugation with any other insecticides (such as pyrethroids, neonicotinoids, carbamates and organophosphates, and others), in the context of a formulation.

### BRIEF DESCRIPTION OF DRAWINGS

For easier understanding of this application, figures are attached in the annex that represent the preferred forms of implementation which nevertheless are not intended to limit the technique disclosed herein.

FIG. 1 shows the structure of 2-(heptadec-10-en-1-yl)-6-hydroxybenzoic acid (1), the synthesis pathway to obtain 2-(heptadec-10-en-1-yl)-6-methoxybenzoic acid (2), 2-(pentadecylamino) benzoic acid (4), 2-(tridecylamino) benzoic acid (5), 2-(dodecylamino) benzoic acid (6a) and 3-(dodecylamino) benzoic acid (6b), from 2-aminobenzoic acid (3a) and 3-aminobenzoic acid (3b), in case of compound (6b).

FIG. 2 shows the viability of insect cells (Sf9 cells) in control conditions (control), with the compound (6a), other related compounds, namely 2-(heptadec-10-en-1-yl) -6-hydroxybenzoic acid (1), 2-(heptadec-10-en-1-yl) -6-methoxybenzoic acid (2), 2-aminobenzoic acid (3a), 3-aminobenzoic acid (3b), 2-(pentadecylamino) benzoic acid (4), 2-(tridecylamino) benzoic acid (5), 3-(dodecylamino) benzoic acid (6b), and in the presence of the commercial insecticide chlorpyrifos (CHPY). Results represent the mean+SEM of at least three independent experiments, each of them performed in triplicate: *p<0.05, **p<0.01, ***p<0.001.

FIG. 3 shows the viability of human keratinocytes in control conditions (control), in the presence of compound (6a) (no toxicity), and in the presence of compound (1) and the commercial insecticide chlorpyrifos, all at 50 μg/mL. Results represent the mean±SEM of at least three independent experiments, each of them performed in triplicate: **<0.01, ***p<0.001.

FIG. 4 shows the caspase-like activity in insect cells in control conditions (basal level, (1), in the presence of compound (6a) (50 μg/mL, 400% increase), in the presence of compound (1) (50 μg/mL, no increase) and in the presence of the commercial insecticide chlorpyrifos (50 μg/mL, no statistically significant changes). Results represent the mean+SEM of at least three independent experiments, each of them performed in triplicate: ***p<0.001.

FIG. 5 discloses the compound of formula (I).

### DESCRIPTION OF EMBODIMENTS

Now, preferred embodiments of the present application will be described in detail with reference to the annexed drawings. However, they are not intended to limit the scope of this application.

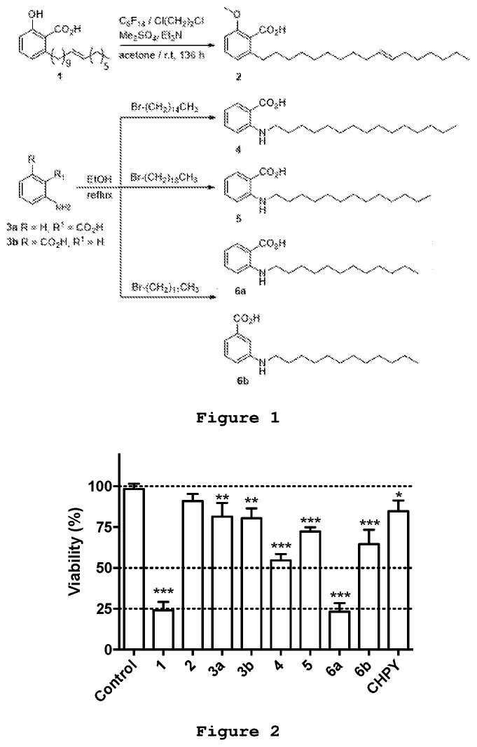

Prior studies showed that natural molecules such as ginkgolic acids (compound (1) in FIG. 1), extracted from Ginkgo biloba leaves, have high toxicity towards insect cells (toxicity towards Sf9 insect cells, causing a loss of ca. 75% of viability at 100 μg/mL). Derivate and improved compound (2) in FIG. 1 had a low yield and had lower activity in Sf9 insect cells.

However, compound (1) exists in very low percentages in its natural source, and a low yield of compound (2) was obtained, therefore obtaining these compounds is extremely time-consuming and expensive. Their low availability makes them inviable to be used as an alternative to currently available insecticides.

The present invention attempts to provide compounds with high toxicity towards insect cells, with high potency, low toxicity to human cells, which can be readily synthesized to be used as insecticides.

Thus, some derivatives of 2-aminobenzoic and 3-aminobenzoic acids possessing side chains with variable number of atoms, derived from compound of Formula (I) were obtained as shown in FIG. 5. Compound (4), compound (5), and compounds (6a, 6b), in FIG. 1, were obtained as examples.

The compound of the present application and its analogues for use as insecticide have the Formula (I):

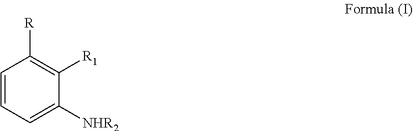

wherein R and R1 are selected from hydrogen or carboxyl group,
when R is hydrogen, R1 is a carboxyl group; and when R is a carboxyl group, R1 is hydrogen,
and R2 is an alkyl group substituted or unsubstituted.

In the present application, the term “alkyl” relates to a linear hydrocarbon group comprising from 12 to 20 carbon atoms, preferably 12 and 15 carbon atoms. The alkyl group can be selected from the dodecyl, tridecyl and pentadecyl groups, substituted or unsubstituted.

FIG. 1 shows a schematic representation of the synthesis pathway to obtain compound 2-(heptadec-10-en-1-yl) -6-methoxybenzoic acid (2) from 2-(heptadec-10-en-1-yl) -6-hydroxybenzoic acid (1) as comparison to the presently disclosed compounds of Formula (I) and its analogues. This same schematic shows the synthesis pathway of the present application in order to obtain compounds such as compounds (6a) and (6b) related to Formula (I).

The method of producing the compound of Formula (I) and its analogues comprises the following steps:
Adding a solution of 1-chloroalkane or of 1-bromoalkane to a solution of 2-aminobenzoic acid or of 3-aminobenzoic acid;
Heating the reaction mixture at a temperature between 50° C. and 70° C., using an alcohol as solvent, and with a final concentration of 0.05 to 0.2 M of the reaction mixture, for a time period between 24 to 64 hours;
Evaporating the solvent under reduced pressure.

The evaporation step can be performed at a temperature between 20 and 70° C. The evaporation step can also be performed at a reduced pressure between 100 and 600 mmHg.

The evaporation step can be followed by a purification step performed, for example, through chromatography, to obtain a pure compound of Formula (I) and its analogues.

In one embodiment the solution of 1-chloroalkane or of 1-bromoalkane is used in a concentration from 0.1 to 0.4 M. In one embodiment the 2-aminobenzoic acid or 3-aminobenzoic acid is used in a concentration from 0.05 to 0.2 M.

In one embodiment methanol or ethanol are used as solvent in a concentration from 80 to 97% (w/w).

The compound (6a) of the present application is more potent than the commercial insecticide chlorpyrifos (FIG. 2), as assessed by using cultivating Spodoptera frugiperda cells in the same cell density, exposing them to either compound (6a) or chlorpyrifos at the same concentration (100 μg/mL) and assessing cell viability after 24 hours using resazurin. The loss of cell viability caused by compound (6a) was more than the triple of that of chlorpyrifos.

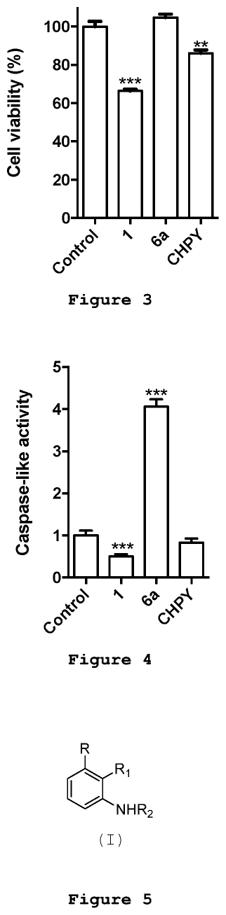

The compound (6a) of the present application has no toxicity in human cells, contrarily to the ginkgolic acid (1), or the synthetic commercial insecticide evaluated as benchmark (chlorpyrifos) (FIG. 3), as assessed by cultivating human keratinocytes in the same cell density, exposing them to either compound (6a) or chlorpyrifos at the same concentration (50 μg/mL) and assessing cell viability after 24 hours using resazurin. Compound (6a) did not cause any loss of cell viability, while chlorpyrifos resulted in ca. 20% of viability loss.

The compound (6a) of the present application presents a mechanism of action distinct of the commercial insecticide chlorpyrifos, which may be of interest as a strategy to overcome pesticide resistances. Specifically, the compound (6a) is capable of activating insect effector caspase DRICE (involved in the process of cell death), while the commercial insecticide is unable to trigger this effect and has no impact in this target (FIG. 4). This was evaluated by incubating cells with the same cellular density with the proluminescent substrate DEVD-aminoluciferin, which becomes fluorescent after cleavage by effector caspases, being detected subsequently in a luminescence detector.

Similarly, to compound (6a) specifically, it is expected that all compounds derived from compound of Formula (I), such as compounds (4), (5) and (6b), present similar potency, no toxicity to human cells, and the same mechanism of action in insect cells. Therefore, the compounds derived from Formula (I) are suitable to be used as insecticide.

In one embodiment, the compound of Formula (I) and its analogues can be incorporated into a formulation for use as insecticide.

### EXAMPLES

Method of producing 2-(dodecylamino) benzoic acid (6a): 1-Bromododecane (0.7 mL, 2.9 mmol) was added to a solution of 2-aminobenzoic acid (3a) (0.2 g; 1.5 mmol). The reaction mixture was refluxed in ethanol (14 mL) for 64 hours and monitored by TLC (DCM/MeOH 9:1). The solvent was evaporated, and the residue obtained was subjected to purification by column chromatography, with DCM/MeOH, as eluent, to afford compound (6a) as a grey solid (0.276 g, 9.0 mmol, 60%). R_{f}=0.33 (DCM/MeOH 9:1).

UV (EtOH)=λmax: 222 (ε616 M⁻¹ cm⁻¹), 249 1664 M⁻¹ cm⁻¹⁾and 355 (ε3224 M⁻¹ cm⁻¹) nm.

FTIR (DCM) νₘₐₓ=2918, 2848, 1669, 1579, 1640, 1574, 1550, 1415, 1255, 1160 cm⁻¹.

1H RMN δ_{H} (Dimethyl Sulfoxide (DMSO), 400 MHz): 7.76 (dd, J =6.4 and 1.6 Hz, 1H, H-6), 7.33 (dt, J=6.8 and 1.6 Hz, 1H, H-4), 6.69 (d, J=8.4 Hz, 1H, H-3), 6.52 (t, J=6.8 Hz, 1H, H-5), 3.13 (t, J=7.2 Hz, 2H, NHCH₂), 1.60-1.53 (m, 2H, NHCH₂CH₂), 1.25-1.35 (m, 18H, 9×CH₂), 0,84 (t, J=6.8 Hz, 3H, CH₃) ppm.

¹³C RMN δc (DMSO, 100.6 MHz): 170.06 (CO₂H), 150.96 (C-2), 134.45 (C-4), 131.67 (C-6), 113.90 (C-5), 111.08 (C-3) , 109.71 (C-1), 41.95 (NHCH₂), 31.28 (CH₂), 29.02 (CH₂), 28.99 (CH₂), 28.95 (CH₂), 28.72 (CH₂), 28.70 (CH₂), 28.56 (CH₂), 26.52 (CH₂), 22.08 (CH₂), 13.93 (CH₃) ppm. HRMS: m/z (ESI-TOF): Found [M+1]⁺: 306.2429; C₁₉H₃₂NO₂ requires [M+1]+: 306.2428.

Method of producing 3-(dodecylamino) benzoic acid 6b: 1-Bromododecane (0.7 mL, 2.9 mmol, 0.5 eq.) was added to a solution of 3-aminobenzoic acid (3b) (0.2 g; 1.5 mmol). The reaction mixture was refluxed in ethanol (14 mL) for 50 hours and monitored by TLC (DCM/MeOH 9:1). The solvent was evaporated, and the residue obtained was subjected to purification by column chromatography, with DCM/MeOH 97:3, as eluent, to afford compound (6b) as a grey solid (0.21 g, 0.61 mmol, 41%).

Rf=0.23 (DCM/MeOH 9:1).

UV (EtOH)=λmax: 227 (ε526 M¹ cm⁻¹), 257 (ε1579 M−1 cm⁻¹⁾ and 340 (ε6380 M⁻¹ cm⁻¹) nm;

FTIR (DCM) νₘₐₓ=2955, 2922, 1679, 1606, 1587 cm⁻¹.

¹H RMN δ_{H} (DMSO, 400 MHz): 7.15 (d, J=7.6 Hz, 3H, H-6) , 7.12-7.08 (m, 2H, H-2 and H-5), 6.6 (d, J=6.8 Hz, 1H, H-4), 5.81 (br s, 1H, NH), 2.96 (t, J=7.2 Hz, 2H, NHCH₂), 1.45-1.50 (m, 2H, NHCH₂CH₂), 1.17-1.29 (m, 18H, 9×CH₂), 0.79 (t, J=7.2 Hz, 3H, CH₃) ppm.

¹³C RMN δ_{c} (DMSO, 100.6 MHz): 166.31 (CO2H), 149.15 (C-3), 130.47 (C-5), 129.05 (C-1), 117.3 (C-6), 116.05 (C-4), 115.90 (C-2), 60.32 (CH₂), 42.69 (NHCH₂), 31.28 (CH₂), 29.03 (CH₂), 29.01 (CH₂), 28.98 (CH₂), 28.84 (CH₂), 28.69 (CH₂), 28.47 (CH₂), 26.60 (CH₂), 22.07 (CH₂), 14.17 (CH₃) ppm. HRMS: m/z (ESI-TOF): Found [M+1]^{+:} 306.2428; C₁₉H₃₂NO₂ requires [M+1]+: 306.2430.

### REFERENCES

1—Insecticides Market By Type, Crop Type, Mode of Application, Formulation and Region—Global Forecast to 2022. Markets and Markets, February, 2017;

2—Europe Insecticides By Type, By Crop Type, By Mode of Application, By Form, And By Region—Industry Analysis, Share, Size, Growth, Trends, and Forecasts (2020-2025), Market Data Forecast, February 2020;3—Eur. JOC, 2007, 24, 4111-4115;

4—Adv. Synth. Catal. 2009, 351, 1671-1676;

5—Pharmaceutical Chemistry Journal, 1971, vol. 5, 1, 7-10.

1. A compound of Formula (I) and its analogues for use as insecticide, wherein Formula (I) is: 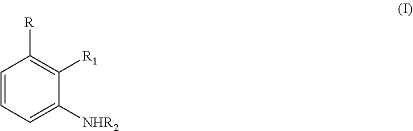
wherein R and R1 are selected from hydrogen or carboxyl group; when R is hydrogen, R1 is a carboxyl group; and when R is a carboxyl group, R1 is hydrogen;
and R2 is an alkyl group substituted or unsubstituted.

2. The compound of Formula (I) and its analogues for use as insecticide according to claim 1, wherein the alkyl group comprises 12 to 20 carbon atoms.

3. The compound of Formula (I) and its analogues for use as insecticide according to claim 2, wherein the alkyl group comprises 12 to 15 carbon atoms.

4. The compound of Formula (I) and its analogues for use as insecticide according to claim 1, wherein the alkyl group is selected from the group consisting of dodecyl, tridecyl and pentadecyl groups, substituted or unsubstituted.

5. The compound Compound of Formula (I) and its analogues for use as insecticide according to claim 1, wherein the compound is a 2-(dodecylamino)benzoic acid of Formula (II):

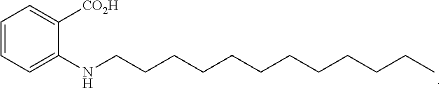 [SMILES: CCCCCCCCCCCCNc1ccccc1C(=O)O]

6. The compound of Formula (I) and its analogues for use as insecticide according to claim 1, wherein the compound is a 3-(dodecylamino)benzoic acid of Formula (III):

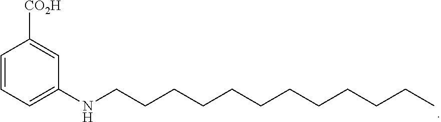 [SMILES: CCCCCCCCCCCCNc1cccc(C(=O)O)c1]

7. A formulation for use as insecticide, comprising the compound of Formula (I) or its analogues described in claim 1.

8. A method of producing the compound of Formula (I) and its analogues described in claim 1, comprising the following steps:
adding a solution of 1-chloroalkene or 1-bromoalkene to a solution of 2-aminobenzoic acid or of 3-aminobenzoic acid to obtain a reaction mixture;
heating the reaction mixture at a temperature between 50° C. and 70° C., using an alcohol as solvent, and with a final concentration of 0.05 to 0.2 M of the reaction mixture, for a time period between 24 to 64 hours;
evaporating the solvent under reduced pressure.

9. The method according to claim 8, wherein the solution of 1-chloroalkene or of 1-bromoalkene has a concentration between 0.1 to 0.4 M.

10. The method according to claim 8, wherein the solution of 2-aminobenzoic acid or of 3-aminobenzoic acid has a concentration between 0.05 to 0.2 M.

11. The method according to claim 8, wherein the alcohol solvent is selected from methanol or ethanol in a concentration from 80 to 97% (w/w).

12. The method according to claim 8, wherein the evaporation step is performed at a temperature between 20 and 70° C.
